(19) United States
(12) Patent Application Publication
(10) Pub. No.: US 2024/0268732 A1
(43) Pub. Date: Aug. 15, 2024

(54) SUBJECT ANALYSIS DEVICE

(71) Applicant: NATSUME RESEARCH INSTITUTE, CO., LTD., Tokyo (JP)

(72) Inventors: Tadashi USUKURA, Tokyo (JP); Hachiro YUGE, Tokyo (JP)

(73) Assignee: NATSUME RESEARCH INSTITUTE, CO., LTD., Tokyo (JP)

(21) Appl. No.: 18/560,108

(22) PCT Filed: May 11, 2022
(86) PCT No.: PCT/JP2022/019904

§ 371 Date: Nov. 10, 2023

(30) Foreign Application Priority Data
May 12, 2021 (JP) 2021-080778

(51) Int. Cl. A61B 5/16; A61B 3/11; A61B 3/113; G06Q 30/0242

(52) U.S. Cl. CPC A61B 5/163; A61B 3/112; A61B 3/113; G06Q 30/0242; A61B 2503/12

(57) ABSTRACT

A method for providing an information to assist validity judgement of a content by measuring a gaze information, a pupil diameter and an attention level of a subject when the subject of a provided content perceives the content is provided. A subject analysis device (1) comprising a content providing unit (5) for providing a content of a still image, a movie or a demonstration to be viewed by a subject; and a gaze position analysis unit (7) for analyzing a gaze position of the subject.

### TECHNICAL FIELD

This invention relates to a subject analysis device.

### BACKGROUND OF THE INVENTION

JP 5445981 B2 describes a device for judging viewer sentiment toward a visual scene.

Conventionally, even when a content such as advertising images is created, it has been difficult to judge the effectiveness of the content as intended by the creator, due to the lack of materials to judge the validity of the content.

### CITATION LIST

### Patent Literature

Patent Literature 1: JP 5445981
Patent Literature 2: JP 6651536

### SUMMARY OF THE INVENTION

### Technical Problem

Therefore, the purpose of this invention is to provide information for assisting judgement of the validity of the content by measuring the gaze position, pupil diameter and attention level of the subject of a provided content when he/she perceives the content.

### Solution to Problem

One invention described in this specification is based on the finding that when the subject of the provided content perceives the content, the effectiveness of the content can be judged by measuring his/her gaze information, pupil diameter and attention level.

One invention described in this specification is a subject analyzing device 1 comprising the following content providing unit 21 and gaze position analysis unit 7.

The content provision unit 21 provides a still image, a movie, or a demonstration content for the subject to view. The gaze position analysis unit 7 analyzes the subject's gaze position.

A preferred example of the present invention is that the gaze position analyzed by the gaze position analysis unit 7 is added to an image information of the content to generate a gaze position-added image information, and an image based on the gaze position-added image information is displayed on an analyzer's display unit 2.

A preferred example of the present invention further comprises an attention point determination unit 9 that determines the subject's attention point in the content image information based on changes in the gaze position analyzed by the gaze position analysis unit.

A preferred example of the present invention further comprises a pupil diameter analyzing unit 11 that analyzes a pupil diameter of the subject.

A preferred example of the present invention is to display the gaze position analyzed by the gaze position analysis unit 7 on the analyzer's display unit 2. Furthermore, based on the pupil diameter of the subject analyzed by the pupil diameter analysis unit 11, it changes the color, size, or shape of the gaze position in the image information of the content displayed on the analyzer's display unit 2.

A preferred example of the present invention further comprises a pupil diameter analysis unit 11 that analyzes the pupil diameter of the subject, and the attention point determination unit 9 determines the attention point of the subject in the content image based on the change in the gaze position analyzed by the gaze position analysis unit 7 and the pupil diameter of the subject analyzed by the pupil diameter analysis unit 11.

### Advantageous Effect of Invention

The present invention can provide information for assisting judgement of the validity of the content by measuring the gaze position, pupil diameter and attention level of the subject of a provided content when he/she perceives the content.

### BRIEF DESCRIPTION OF THE DRAWINGS

FIG. 1 shows a block diagram of a subject analysis device of the present invention.

FIG. 2 shows a drawing showing an example of a screen of a pupil analysis result player.

FIG. 3 shows a drawing showing an example of a pupil diameter graph displayed on the screen of the pupil analysis result player.

FIG. 4 shows a drawing showing an example of an attention graph displayed on the screen of the pupil analysis result player.

FIG. 5 shows a drawing showing an example of a file content output in the present invention.

FIG. 6 shows a drawing showing an example of a file content output in the present invention.

FIG. 7 shows a flow chart showing an example of a process performed in the present invention.

### DETAILED DESCRIPTION OF AN EXEMPLARY IMPLEMENTATION

An embodiment of the present invention is described hereinafter with reference to the drawings. The present invention is not limited to the embodiment described below, but includes those that have been modified from the following embodiment as appropriate within the scope obvious to those skilled in the art.

### <Description of Each Element>

A subject analysis device 1 described below may basically comprise hardware such as a control arithmetic unit, a memory unit, an input unit, and an output unit. The control arithmetic unit executes a predetermined arithmetic process according to a program stored in the memory unit based on information input from the input unit, and controls the output unit while writing and reading results of the arithmetic to and from the memory unit as appropriate. Examples of the control arithmetic device are processors such as CPU and GPU. The storage function of the storage unit can be realized by non-volatile memory, such as HDD and SSD, and the memory function of the storage unit can be realized by volatile memory, such as RAM and DRAM. Examples of the input device are communication modules for receiving information via a network and operation modules such as a mouse and a keyboard. Examples of the output device are communication modules for transmitting information via the network, displays, speakers, etc.

For example, the storage unit may store a control program or various information. When predetermined information is input from the input device, the control unit reads out the control program stored in the memory unit. The control unit then reads the information stored in the memory unit and sends it to the arithmetic unit as appropriate. The control unit also sends the input information to the arithmetic unit as appropriate. The arithmetic unit performs arithmetic processing using the received information and stores it in the memory unit. The control unit reads out the arithmetic results stored in the memory unit and outputs them from the output unit. In this way, various processes are executed. The various processing is executed by the various means.

In the present invention, first, the subject is asked to perceive a video, a still image, or an actual content, and the eyeball information at that time is obtained and analyzed. At the same time, the subject's perceived content is captured as a video at the camera angle of the subject's gaze. Markers located at the subject's gaze position are added in the above image information at each time interval, and thereby a pupil analysis result video is processed and generated. The video is then played back and displayed on an analyzer's display unit 2, etc., by user operation. Furthermore, based on the above information, a content validity judging unit 23 performs a validity judgement of the content.

### (Subject Analysis Device 1)

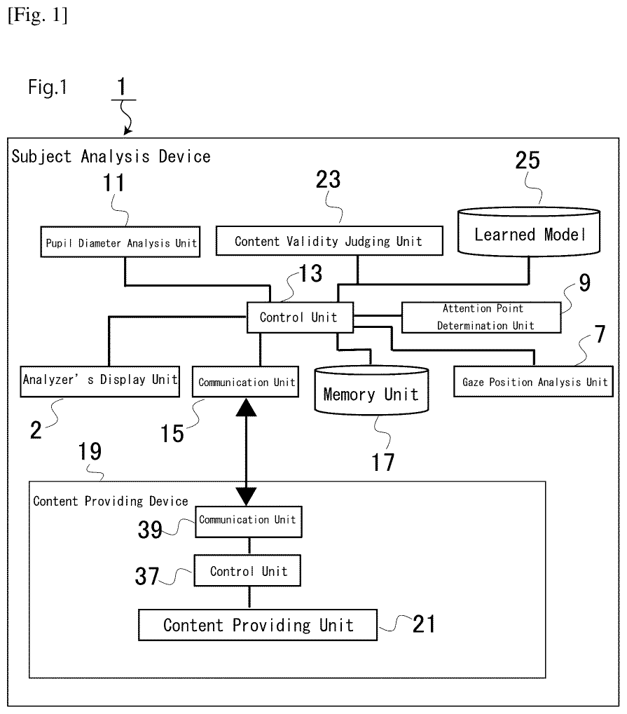

FIG. 1 shows a block diagram of the subject analysis device 1 of the present invention.

The subject analysis device 1 comprises the analyzer's display unit 2, a gaze position analysis unit 7, a control unit 13, and a communication unit 15. Furthermore, it is preferable that the subject analysis device 1 comprises an attention point determination unit 9 and a pupil diameter analysis unit 11. It is also preferable that the subject analysis device 1 has a content providing device 19. The subject analysis device 1 may be provided with the content validity judging unit 23.

The subject analysis device 1 is a device for analyzing the gaze position and changes in gaze position of the subject viewing a still image or video, etc.

It is preferable that a camera or an external device is connected to the subject analysis device 1 to obtain eyeball information and visual information of the subject.

### (Analyzer's Display Unit 2)

The analyzer's display unit 2 is a liquid crystal display or the like, and displays various information of a pupil analysis result playback player 4 etc. Note that if the content provided by the content providing unit 21 described below is video or still image, and the content providing unit 21 is a display, etc., the analyzer's display section 2 may be the same as the content providing unit 21.

### (Pupil Analysis Result Playback Player 4)

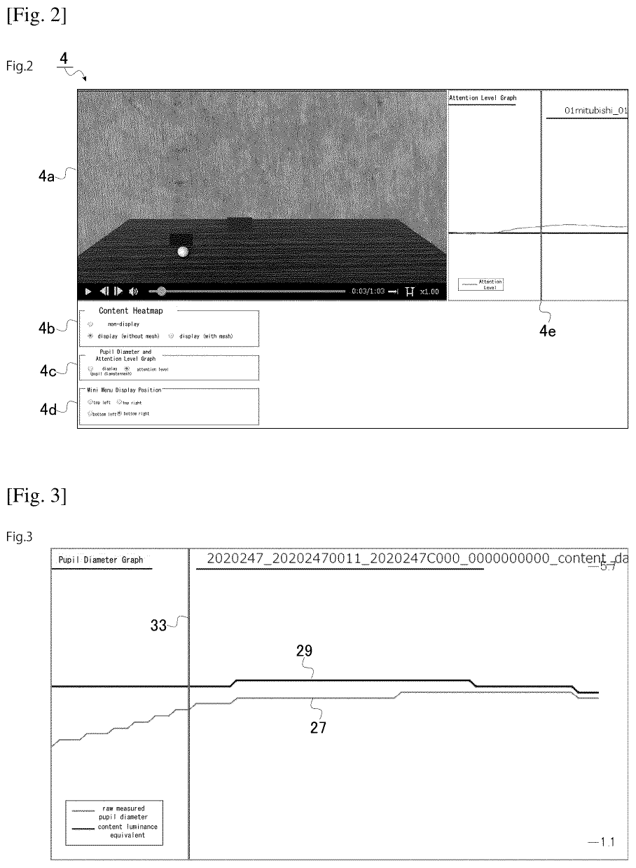

FIG. 2 shows the content of a pupil analysis result video display player 4 of the subject analysis device 1.

The pupil analysis result video display player 4 is an element for displaying the pupil analysis results of the subject sensing the content.

The pupil analysis result video display player 4 may run on the web, or it may run offline in a terminal such as a PC. As shown in FIG. 2, the pupil analysis result video player 4 may have, for example, an analysis result video display field 4a for displaying the subject's attention level and pupil analysis results.

The pupil analysis result video display player 4 may be provided with a content heat map setting field 4d, a pupil diameter and attention level graph display setting field 4c, a mini menu display position setting field 4d, and a pupil diameter and attention level graph display field 4e.

The analysis result movie display field 4a is a part that displays images corresponding to the gaze position and the attention level of the subject of the content. Specifically, for example, the image displayed in the analysis result video display field 4a is the content perceived by the subject, as a basic image, to which information such as the subject's gaze position and the attention point etc. is added as follows. The subject's gaze position while perceiving the content etc. is added and displayed on the basic image as a predetermined heat map marker.

In other words, the actual subject's gaze position while perceiving the content corresponds to the position of the heat map markers in the above image information. The markers may change color, size or shape according to the subject's attention level.

At the bottom of the analysis result video display field 4a, for example, a video playback button, a rewind button, and a fast-forward button may be provided. In addition, a mini menu for setting the playback position and playback speed may also be displayed in the analysis result video display field 4a.

The pupil analysis result video display player 4 may be provided with a heat map setting field 4b, such as a radio box, where the user (analyzer) can select whether or not to display markers such as the heat map marker etc. in the analysis result video. For example, if the non-display mode in the heat map setting field 4b is selected and the play button is pressed, the video is played without the markers of interest in the above analysis result video. When the display mode in the content heat map setting field is selected and the play button is pressed, the video is played with the markers of the attention point displayed in the above analysis result video. In addition, when markers are displayed on the analysis result video, mesh lines may be displayed on the video.

The pupil analysis result video display player 4 may be provided with a pupil diameter and attention level graph selection field 4c, which has a radio box that allows the user to select which of the above graphs to display or not.

If the pupil diameter radio box is selected, the pupil diameter graph is displayed in the pupil diameter and attention level graph display field 4e, and if the attention level radio box is selected, the attention level graph is displayed in the pupil diameter and attention level graph display field 4e.

The pupil analysis result video display player 4 may have the pupil diameter and attention level graph display field 4d that displays a pupil diameter graph representing the subject's pupil diameter and an attention level graph representing the subject's attention level. The graph representing the subject's pupil diameter and the graph representing the subject's attention level may be displayed in a manner where only one of the two graphs is displayed, or where both graphs are displayed.

The method for measuring attention level, the pupil diameter graph and the attention level graph are described below.

In addition to this, the pupil analysis result video display player 4 may be provided with the mini menu display position setting field 4d.

The mini menu display position can be set in the mini menu display position setting field 4d.

### (Pupil Diameter, Pupil Diameter Graph)

The subject analysis device 1 receives the pupil diameter information of the subject from an external device having a camera or the like. The method of measuring pupil diameter used in the present invention may be a method such as that shown in JP 6651536 B2.

According to the invention of JP 6651536 B2, the pupil diameter can be measured without the effects of light and dark, respiration and pulse, and the viewer's emotion can be judged more accurately.

Note that in the present invention, a pupil diameter that can be measured by any other known method may be used as long as the pupil diameter can be measured appropriately.

The graph of FIG. 3 is a graph representing the time change of pupil diameter when the subject perceives the content. This graph is displayed in the pupil diameter and attention level graph display field 3e in the display unit 3.

The horizontal direction of this graph indicates how far away in time the analyzed result video is from the time position (a time position line during video playback 33) during playback.

In this graph, the time position line during video playback 33 as the vertical line indicates the position (time) of the pupil analysis result video that is displayed on the analyzer display unit 2 and is being played. On the graph, the left side of the time position line during video playback 33 is the information about the attention level at the time position that has already been played by the pupil analysis result video playback player 4, and the right side is the information about the attention level at the time position that has not been played by the pupil analysis result playback player 4.

Further, the vertical direction of this graph (the upward direction in FIG. 3 is the positive direction) represents the pupil diameter and a size of content luminance equivalent.

In this graph, the raw measured pupil diameter 27, which is a horizontal line, is a line formed by a set of points representing the size of the pupil diameter at each playback time position of the subject.

In this graph, the horizontal content luminance equivalent 29 is a line formed by a set of points representing the size of the pupil diameter assumed from the content luminance at each playback time position of the subject.

In other words, the raw measured pupil diameter 27 and the content luminance equivalent 29 will shift to the left direction in the graph as the playback position (time) of the analyzed video advances.

In addition to the above, the specification of the pupil diameter graph may be as follows.

The specification may be that the raw measured pupil diameter 27 and the content luminance equivalent 29 for the entire time of the analysis result video are first fixedly displayed before video playback, and the time position line 33 shifts during video playback in response to the position (time) during video playback.

### (Attention Level and Attention Level Graph)

The attention level is a measure of the level to which the subject pays attention to the object. For example, the attention level can be measured by the method described in JP 6651536 B2 above.

Note that in the present invention, an attention level that can be measured by any other known method may be used as long as the attention level can be measured appropriately.

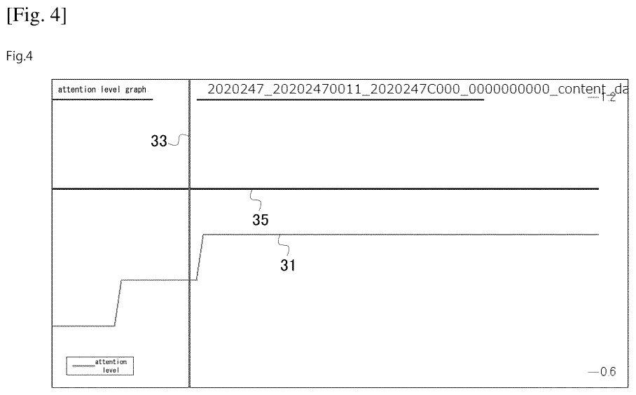

The graph of FIG. 4 (attention level graph) is a graph that shows the subject's time change of attention level when the content is perceived. This graph is displayed in the pupil diameter and attention level graph display field 4e of the pupil analysis result playback player 4.

The horizontal direction of this graph indicates how far away in time the analyzed result video is from the time position (a time position line during video playback 33) during playback.

In this graph, the time position line during video playback 33, which is the vertical line, indicates the position (time position) of the pupil analysis result video that is displayed on the pupil analysis result playback player 4 and is being played. On the graph, the left side of the time position line during video playback 33 is the information about the attention level at the time position that has already been played by the pupil analysis result video playback player 4, and the right side is the information about the attention level at the time position that has not been played by the pupil analysis result playback player 4.

Further, the vertical direction of this graph (the upward direction in FIG. 4 is the positive direction) represents the degree of the attention level.

In this graph, the horizontal attention level 31 is a line formed by a set of points representing the degree of the attention level at each playback time position of the subject.

In other words, the line of the attention level 31 will shift to the left direction in the graph as the playback position of the analyzed video advances.

In this graph, a horizontal attention level reference line 35 is a reference line located at a height equivalent to the attention level value (preferably greater than 1.0) and is fixedly displayed on the graph.

In addition to the above, the specification of the attention level graph may be as follows.

The specification may be that the attention level 31 and the attention level reference line 35 for the entire time of the analysis result video are first fixedly displayed before the video playback, and the time position line during video playback 33 shifts in response to the position (time) during the video playback.

### (Content Providing Device 19)

The content providing device 19 is a device for providing the content to the subject. The content providing device 19 comprises a content providing unit 21, a control unit 37 and a communication unit 39.

### (Content Providing Unit 21)

The content providing unit 21 is a unit for providing the content to the subject. The content provided by the content providing unit 21 is those that appeal to the visual sense.

For example, the content providing unit 21 may be a display or a monitor that provides movies, etc. Note that when the content provided by the content providing unit 21 is a video etc., the content providing unit 21 may be the same device as the analyst's display unit 2 etc. Examples of the content provided by the content providing unit 21 are video advertisements, still image advertisements, and the like. Other examples of content include advertisement of demonstrative performance by actual products and performers.

### (Gaze Position Analysis Unit 7)

The gaze position analysis unit 7 is an element for analyzing the subject's viewpoint position based on the image of the content perceived by the subject and the subject's eye movement information captured by an external camera etc. The gaze position analysis unit 7 acquires the eye movement image captured by the external camera.

If the content perceived by the subject is image information such as a movie, a still image etc., the gaze position analysis unit 7 acquires that image information from the content providing device etc., as an image information of the content.

On the other hand, if the content perceived by the subject is not the image information but a demonstration as described above, the image of the demonstration is captured by an external camera, and the gaze position analysis unit 7 acquires that image information as the image information of the content. Note that the image information of the content shall include the image information from the camera angle of the subject's viewpoint.

The image information of the content referred to below shall represent the above image information.

Then, the gaze position analysis unit 7 identifies the subject's viewing position in the image from the image information of the content and eye movement images, and stores its information in the memory unit 17.

Note that the method of analyzing the viewpoint position used in the gaze position analysis unit 7 may be any known method as long as it can identify the viewpoint position in the image of the content perceived by the subject.

### (Attention Point Determination Unit 11)

The attention point determination unit 11 is an element for determining which part of the content image the subject perceiving the content is paying attention to. For example, the attention point determination unit 11 receives the image information of the content perceived by the subject and the eye movement information of the subject etc., similar to the method of the gaze position analysis unit 7 described above. Then, the information of the screen part of the image screen and the subject's attention level of the part are stored in the memory unit 17.

By using these information, the subject's gaze position represented by various markers described below can be added to the pupil analysis result video displayed in the pupil analysis result playback player 4.

### (Displaying of Gaze Position)

The subject analysis device 1 generates display mode information of the markers of the gaze position to be displayed on the analysis result video based on the information obtained by the gaze position analysis unit 7, etc, described later. The above information is then added to the image information of the content to generate the gaze position-added image information.

Then, an image based on the gaze position-added image information is displayed on the analyzer's display unit 2.

This makes it possible to display the markers of the above-mentioned gaze position at the subject's gaze position in the image of the content perceived by the subject.

For example, based on the playback position time of the video and the attention level at each gaze position, the color of the marker at the part in the analyzed result video is set. If the attention level is above a certain value, the part of the marker is colored red, and if it is below that value, it is colored blue.

The display mode of the marker at the gaze position may not only change color, but also change the size and shape of the marker.

### (Pupil Diameter Analysis Unit 13)

The pupil diameter analysis unit 13 is an element for analyzing the pupil diameter of the subject. The pupil diameter analysis unit 13 acquires the subject's eye information obtained from an external camera etc.

The method of measuring the pupil diameter in the present invention assumes the method shown in JP 6651536 B2, as described above (Pupil Diameter, Pupil Diameter Graph). The method for measuring the pupil diameter of the present invention may be any other known method as long as it can accurately measure the pupil diameter.

### (Content Validity Judging Unit 23)

The subject analysis device 1 may comprises the content validity judging unit 23. The content validity judging unit 23 is an element for judging the validity of the content provided to the subject. The result of the content validity judgment may be displayed on the analyzer's display unit 2, etc., or output as a report file.

Here, the validity of the content is an index that indicates, for example, how much the video advertisement can contribute to the sales of a certain product when the provided content is a video advertisement of a certain product. In other words, in the above example, the content validity judging unit can predict the contribution of the provided content to the sales of the product based on the distribution of the gaze position and the attention level in the content video.

The diagnostic process of the content validity judging unit 23 may be performed using the learned model 25. The learned model 25 is model data whose parameters (so-called “weights”) have been adjusted by machine learning on the biological data of many subjects.

For example, a learned model 25 is created by implementing machine learning, such as deep learning, using the content provided to a large number of subjects, their gaze position, the pupil diameter, the attention level and the validity of the content as teacher data. In this case, by referring to this learned model 25 as input values for the content, the gaze position, the pupil diameter and the attention level provided to a subject, the results of the content validity judging can be obtained as output values corresponding to those input values. The subject analysis device 1 may have such a learned model in advance. However, this learned model 25 is not an essential element.

### (Control Unit 13, Control Unit 37)

The control unit 13 is an element for giving processing instructions to each element of the subject analysis device 1. It is also an element for accessing the memory unit, etc., for referencing, registering, updating, etc., data.

The control unit 37 is an element for giving processing instructions to each element of the content providing device.

### (Communication Unit 15, Communication Unit 39)

The communication unit 15 has functions for the subject analysis device 1 to send and receive information to and from the content providing device 19 etc. The communication unit 15 is an element that performs communication in accordance with optional communication standard.

The communication unit 39 has functions for the content providing device 19 to send and receive information to and from the subject analysis device 1 etc. The communication unit 39 is an element that performs communication in accordance with optional communication standard.

### (Analysis Result Output File)

The measurement results of the subject's pupil diameter and the attention level by the subject analysis device described above are output as a report file. The output destination may be in the subject analysis device or in an external device.

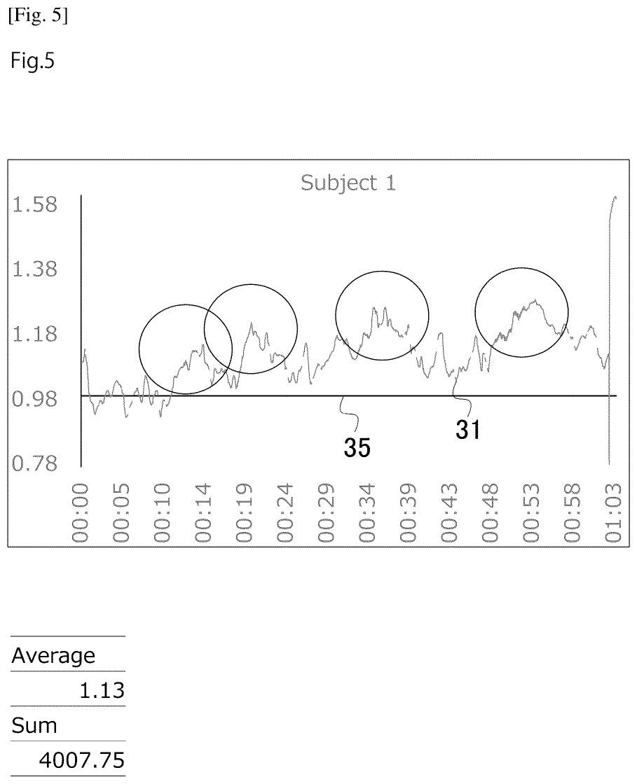

FIG. 5 shows the graph data of the output file data, which shows the time trends of each subject's attention level and its average value.

The horizontal axis of the graph in FIG. 5 is the time axis and the vertical axis is the subject's attention level.

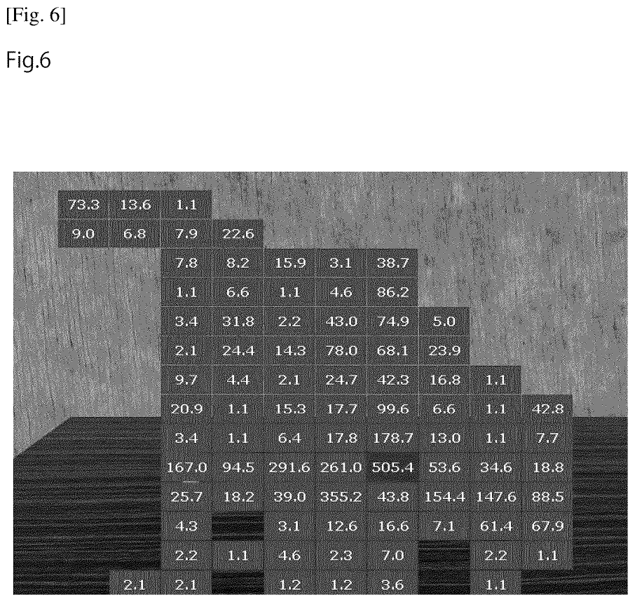

FIG. 6 shows the attention level total number distribution data for each subject in the output file data. The above-mentioned attention level total number distribution data is a illustration of data for aggregating the attention level total number distribution for each subject among the output file data.

In addition to the above files, a file may also be output containing information of the standard deviation indicating the variation of attention level among more than one subject, or information of the standard deviation indicating the variation of attention level during the measurement of one subject.

### <Operation of Information Processing in Subject Analysis Device 1>

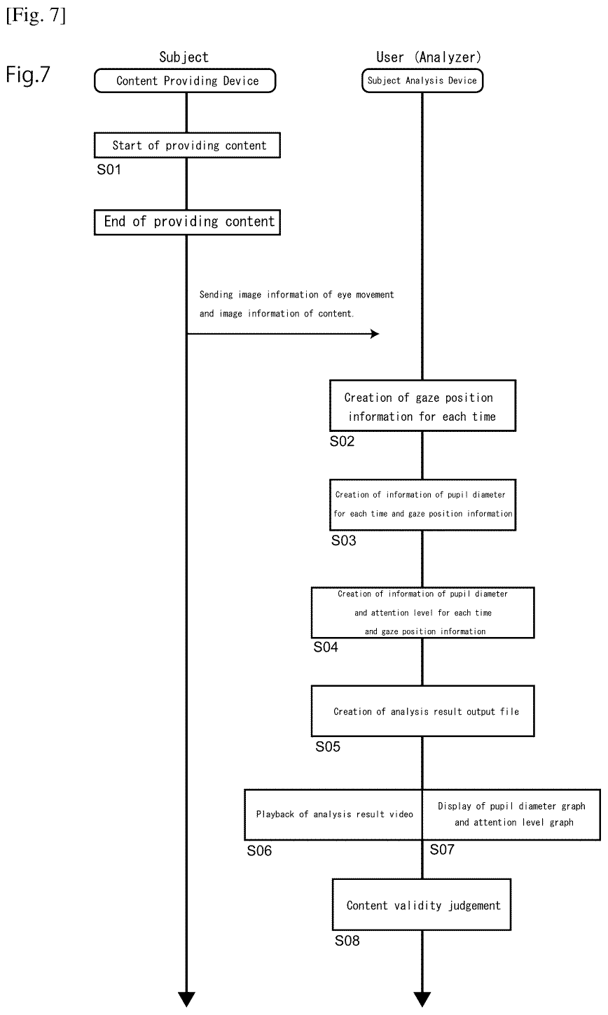

The processing of the information processing system according to an example of an embodiment of the present invention is described below. FIG. 7 is a flow diagram showing an example of a process executed in the present invention.

Note that the processing flow described below is an example of internal processing to realize the subject analysis device 1 of the present invention, and internal processing that can be used for the subject analysis device 1 of the present invention is not limited to the following example.

(1. Acquisition of Visual Information and Image Information of the Subject, Creation of Video Information for Analysis)

When the user (analyst etc.) presses the provision start button of the content providing device, provision of the content (sample video playback etc.) to the subject is started (S01 Provision of content).

At the same time as the provision of the content S01, image information of the subject's eye movement and image information of the content are acquired by a known method.

The information of subject's eye movement and the image information of the content are then sent to the gaze position analysis unit 7 using a predetermined method.

Based on these information, the gaze position analysis unit 7 creates gaze position information for each time in the image of the subject's content. The image information of the content, the gaze position information and the eye movement information may be stored in the memory section 17 etc. (S02 Creation of gaze position information).

The above image information of the content, the gaze position information and the eye movement information are then sent to the pupil diameter analysis unit 11 by a predetermined method. The pupil diameter analysis unit 11 measures the pupil diameter using a known method based on the above-mentioned eye movement, the content image and its luminance etc. The pupil diameter information is created for each time and each gaze position information in the image information of the subject's content. The above information is stored in the memory unit 17, etc. (S03 Creation of pupil diameter information).

Thereafter, the above content image information, the gaze position information and the eye movement information are sent to the attention point determination unit 9 by a predetermined method. The attention point determination unit 9 measures the attention level using a known method based on the above-mentioned eye movement, the content image information and its luminance, etc. Then, information of the pupil diameter and the attention level are created for each time and each gaze position information in the image information of the subject's content. (S04: Creation of attention level information).

Through the above process from S01 to S04, information for the subject's pupil analysis result movie are created. Based on these information, the above-mentioned analysis result output file is created (S05 Creation of analysis result output file).

### (2. Display of Pupil Analysis Result)

When the user (analyst) presses the video play button of the pupil analysis result playback player 4, the gaze position-added image information is created based on information created through S01 to S04 above in the analysis result video display field 4a of the pupil analysis result playback player 4.

Then, the analysis result video, which is the image based on the gaze position-added image information, is played back and displayed (S06 Playback of analysis result video). At that time, a pupil diameter graph and an attention level graph are displayed at the same time as the above video playback, based on the selection made in the pupil diameter and attention level graph display setting field 4c of the pupil analysis result playback player 4 (S07 Display of graph). In addition, the result of the content validity judgment may be displayed and output according to the user's (analyst's) operation (S08 Content validity judgement).

The above description of embodiments of the present invention has been given in this specification with reference to the drawings in order to realize the content of the present invention. However, the present invention is not limited to the above embodiments, but encompasses modified embodiments and improved embodiments that are obvious to those skilled in the art based on the matters described in this specification.

### INDUSTRIAL APPLICABILITY

This invention can be used in the field of information analysis.

### LIST OF REFERENCE NUMERALS

1 subject analysis device
2 analyzer's display
4 pupil analysis result playback player
4a analysis result video display field
4b heatmap display setting field
4c pupil diameter and attention level selection field
4d mini menu display position setting unit
4e pupil diameter and attention level graph display field
7 gaze position analysis unit
9 attention point determination unit
11 pupil diameter analysis unit
13 control unit
15 communication unit
17 memory unit
19 content providing device
21 content providing unit
23 content validity judging unit
25 learned model
27 raw measured pupil diameter
29 content luminance equivalent
31 attention level
33 time position line during video playback
35 attention level reference line
37 control unit
39 communication unit

What is claimed:

1. A subject analysis device (1) comprising:
a content providing unit (21) for providing a content of a still image, a movie or a demonstration to be viewed by a subject; and a gaze position analysis unit (7) for analyzing a gaze position of the subject.

2. The subject analysis device (1) according to claim 1, wherein
the gaze position analyzed by the gaze position analysis unit (7) is added to an image information of the content to generate a gaze position-added image information, and an image based on the gaze position-added image information is displayed on an analyzer's display unit (2).

3. The subject analysis device (1) according to claim 1, wherein
the subject analysis device (1) further comprises an attention point determination unit (9) that determines an attention point of the subject in the image information of the content based on a change in the gaze position analyzed by the gaze position analysis unit (7).

4. The subject analysis device (1) according to claim 1, wherein
the subject analysis device (1) further comprises a pupil diameter analysis unit (11) that analyzes a pupil diameter of the subject.

5. The subject analysis device (1) according to claim 4, wherein
the subject analysis device (1) displays the gaze position analyzed by the gaze position analysis unit (7) on the analyzer's display unit (2), and changes a color, a size, or a shape of the gaze position displayed on the analyzer's display unit (2) based on the pupil diameter of the subject analyzed by the pupil diameter analysis unit (11).

6. The subject analysis device (1) according to claim 3, wherein
the subject analysis device (1) further comprises a pupil diameter analysis unit (11) that analyzes a pupil diameter of the subject, and wherein
the attention point determination unit (9) determines the attention point of the subject in the displayed image based on the change in the gaze position analyzed by the gaze position analysis unit (7) and the pupil diameter of the subject analyzed by the pupil diameter analysis unit (11).
